(19) United States
(12) Patent Application Publication
(10) Pub. No.: US 2004/0110990 A1
(43) Pub. Date: Jun. 10, 2004

(54) Catalytic reduction of nitriles to aldehydes

(76) Inventors: Markus Eckert, Shanghai (CN); Friedrich Durholz, Remscheid (DE)

Correspondence Address: BAYER CHEMICALS CORPORATION, PATENT DEPARTMENT, 100 BAYER ROAD, PITTSBURGH, PA, 15205-9741, US

(21) Appl. No.: 10/641,199

(22) Filed: Aug. 14, 2003

(30) Foreign Application Priority Data
Aug. 14, 2002 (DE) 10237196.2

(51) Int. Cl. C07F 9/02; C07C 45/90

(52) U.S. Cl. 568/11; 568/426

(57) ABSTRACT

The invention relates to a process for catalytically reducing substituted benzonitriles to substituted benzaldehydes in the presence of aqueous formic acid, of a nickel- and aluminium-containing catalyst and hydrogen.

### BACKGROUND OF THE INVENTION

1. Field of the Invention

The invention relates to a process for catalytically reducing substituted benzonitriles to substituted benzaldehydes in the presence of water, acid, a nickel- and aluminium-containing catalyst and hydrogen.

2. Brief Description of the Prior Art

Substituted benzaldehydes have great industrial importance as fine chemicals and active ingredient intermediates.

Substituted benzaldehydes are frequently prepared by the selective reduction of the corresponding benzoic acid derivatives, usually using complex metal hydrides or using a palladium catalyst by the Rosenmund method. These processes have the disadvantage that they use either expensive hydride sources or expensive catalysts. In addition, the benzoic acid derivatives used are usually prepared from the corresponding benzonitriles by hydrolysis, so that it would be advantageous to convert the benzonitrile directly to the benzaldehyde.

U.S. Pat. No. 5,124,487 states that p-trifluoromethylbenzaldehydes can be prepared from the p-trifluoromethylbenzonitriles by a catalytic reduction with hydrogen in aqueous formic acid in the presence of a nickel/aluminium catalyst. However, the process is restricted to specific substitution patterns and in particular to the presence of a strongly electron-withdrawing trifluoromethyl group in the para-position.

### SUMMARY OF THE INVENTION

Surprisingly, a process has now been found for preparing benzaldehydes of the formula (I)

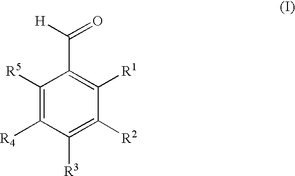

where

R¹, R², R⁴ and R⁵ are each independently hydrogen, fluorine, free or protected formyl, C₁-C₁₂-alkyl, C₁-C₁₂-alkoxy, C₁-C₁₂-haloalkyl, C₁-C₁₂-haloalkoxy, C₄-C₁₄-aryl, C₅-C₁₅-arylalkyl, —PO—[(C₁-C₈)-alkyl]₂, —PO—[(C₄-C₁₄)-aryl]₂, —PO—[(C₁-C₈)-alkyl)(C₄-C₁₄)-aryl)], tri(C₁-C₈-alkyl)siloxyl

or radicals of the formula (II)

A—CO—B (II)

where, each independently,

A is absent or is a C₁-C₈-alkylene radical and

B is R⁶, OR⁶, NHR⁷ or N(R⁷)₂,

where R⁶ is C₁-C₈-alkyl, C₅-C₁₅-arylalkyl, C₁-C₈-haloalkyl or C₄-C₁₄-aryl and

R⁷ is in each case independently C₁-C₈-alkyl, C₅-C₁₅-arylalkyl or C₅-C₁₄-aryl, or N(R⁷)₂ together is a cyclic amino radical,

or radicals of the formulae (IIIa-e)

A—E (IIIa)

A—SO₂—B (IIIb)

A—SO₂R⁶ (IIIc)

A—SO₃W (IIId)

A—COW (IIIe)

where

A, B and R⁶ are as defined above and

W is OH or NH₂, and

R³ is hydrogen, fluorine, chlorine or bromine,

which is characterized in that compounds of the formula (IV)

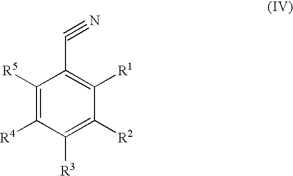

where

R¹, R², R³, R⁴ and R⁵ are as defined under formula (I) are reacted

with hydrogen in the presence of water and

in the presence of acid or acidic salts, the acids or acidic salts having a pK_{A} value of from 1 to 6 based on an aqueous basis system at 25° C., and

in the presence of a nickel- and aluminium-containing catalyst.

Alkyl and alkoxy are in each case independently a straight-chain, cyclic, branched or unbranched alkyl and alkoxy radical respectively. The same applies to the nonaromatic moiety of an arylalkyl radical.

C₁-C₄-Alkyl is, for example, methyl, ethyl, n-propyl, isopropyl, n-butyl, sec-butyl and tert-butyl, C₁-C₈-alkyl is additionally, for example, n-pentyl, 1-methylbutyl, neopentyl, cyclohexyl, cyclopentyl, n-hexyl, n-heptyl and n-octyl, and C₁-C₁₂-alkyl is further additionally, for example, n-nonyl, n-decyl and n-dodecyl.

C₁-C₄-Alkoxy is, for example, methoxy, ethoxy, n-propoxy, isopropoxy, n-butoxy, sec-butoxy and tert-butoxy, C₁-C₈-alkoxy is additionally n-pentoxy, cyclohexoxy, cyclopentoxy, n-hexoxy and n-octoxy, and C₁-C₁₂-alkoxy is further additionally, for example, n-decoxy and n-dodecoxy.

Haloalkyl and haloalkoxy are in each case independently a straight-chain, cyclic, branched or unbranched alkyl radical and alkoxy radical respectively, each of which is singly, multiply or fully substituted by bromine, chlorine and/or fluorine atoms, preferably by fluorine atoms.

For example, C₁-C₁₂-haloalkyl in all contexts is preferably trifluoromethyl, 2,2,2-trifluoroethyl, pentafluoroethyl, nonafluorobutyl, heptafluoroisopropyl, perfluorooctyl and perfluorododecyl.

For example, C₁-C₁₂-haloalkoxy in all contexts is preferably trifluoromethoxy, 2,2,2-trifluoroethoxy, heptafluoroisopropoxy and pentafluoroethoxy.

Aryl is in each case independently a heteroaromatic radical having from 4 to 14 framework carbon atoms of which no, one, two or three framework carbon atoms per cycle, but at least one framework carbon atom in the entire molecule, may be substituted by heteroatoms selected from the group of nitrogen, sulphur or oxygen, but is preferably a carbocyclic aromatic radical having from 6 to 14 framework carbon atoms.

Examples of carbocyclic aromatic radicals having from 6 to 14 framework carbon atoms are, for example, phenyl and naphthyl, and heteroaromatic radicals having from 4 to 14 framework carbon atoms of which no, one, two or three framework carbon atoms per cycle, but at least one framework carbon atom in the entire molecule, may be substituted by heteroatoms selected from the group of nitrogen, sulphur or oxygen are, for example, pyridinyl, benzofuranyl, dibenzofuranyl or quinolinyl.

The carbocyclic aromatic radical or heteroaromatic radical may also be substituted by up to five identical or different substituents per cycle which are selected from the group of chlorine, fluorine, C₁-C₁₂-alkyl, C₁-C₁₂-alkoxy, C₁-C₁₂-haloalkyl, C₁-C₁₂-haloalkoxy, di(C₁-C₈-alkyl)amino, COO(C₁-C₈-alkyl), CON(C₁-C₈-alkyl)₂, COO(C₁-C₈-alkyl), COO(C₄-C₁₄-aryl), CO(C₁-C₈-alkyl), C₅-C₁₅-arylalkyl or tri(C₁-C₆-alkyl)siloxyl.

C₄-C₁₄-Aryl is, for example and with preference, phenyl, o-, p-, m-tolyl, o-, p-, m-anisyl, o-, p-, m-fluorophenyl, o-, p-, m-chlorophenyl, o-, p-, m-trifluoromethyl-phenyl, o-, p-, m-nitrophenyl and 2-, 3- and 4-pyridyl.

Arylalkyl is in each case independently a straight-chain, cyclic, branched or unbranched alkyl radical as defmed above which may be singly, multiply or fully substituted by aryl radicals as defined above.

C₅-C₁₅-Arylalkyl is, for example and with preference, benzyl or (R)- or (S)-1-phenylethyl.

Protected formyl is a formyl radical which is protected by conversion to an aminal, acetal or mixed aminalacetal, and the aminals, acetals and mixed aminalacetals may be acyclic or cyclic.

For example and with preference, protected formyl is a 1,1-(2,5-dioxy)cyclo-pentylene radical.

The preferred substituted patterns for compounds of the formulae (I) and (II) are defined hereinbelow:

R¹, R², R⁴ and R⁵ are preferably each independently hydrogen, fluorine, C₁-C₄-alkyl, C₁-C₄-alkoxy, C₁-C₄-haloalkyl, C₁-C₄-haloalkoxy or N(R⁷)₂ where R⁷ is in each case independently, but preferably identically, methyl or ethyl.

R¹, R², R⁴ and R⁵ are more preferably each independently hydrogen, fluorine, trifluoromethyl and pentafluoroethyl, most preferably hydrogen or trifluoromethyl.

R³ is preferably hydrogen, fluorine or chlorine.

Particularly preferred compounds of the formula (IV) are:

4-chlorobenzonitrile, 3-trifluoromethylbenzonitrile, 3,5-bis(trifluoromethyl)benzo-nitrile and 3,5-difluorobenzonitrile.

The process according to the invention is carried out in the presence of water and in the presence of acid or acidic salts, the acids or acidic salts having a pK_{A} value of from 1 to 6 based on an aqueous basis system at 25° C. The weight ratio of acid and/or acidic salt to water is preferably from 1:10 to 10:1, more preferably from 1:1 to 4:1.

Preferred acids are carboxylic acids, in particular formic acid, acetic acid or propionic acid, and greater preference is given to formic acid and acetic acid.

The process according to the invention is carried out in the presence of a nickel- and aluminium-containing catalyst.

The weight ratio of nickel to aluminium may, for example, be from 1:20 to 20:1, preferably from 1:5 to 5:1 and more preferably from 0.9:1 to 1.1:1.

The nickel- and aluminium-containing catalyst can be used either as a mixture of the metals or in the form of an alloy, and preference is given to the use as an alloy.

Preference is given to using the nickel- and aluminium-containing catalyst in finely divided form, for example as a powder or dust or in the form of pieces.

The nickel- and aluminium-containing catalyst may further comprise one or more metals from the group of chromium, rhenium, iron, cobalt, molybdenum and copper.

The amount of the nickel- and aluminium-containing catalyst may, for example, be from 0.5 to 50 per cent by weight, based on the compound of the formula (IV) used, preferably from 3 to 30 per cent by weight and more preferably from 5 to 20 per cent by weight. Larger amounts of catalyst are possible but uneconomic.

According to the invention, compounds of the formula (IV) are reacted with hydrogen.

The hydrogen pressure may, for example, be from 0.2 to 100 bar, preferably from 1 to 20 bar, more preferably from 2 to 10 bar.

The reaction temperature may, for example, be from 20° C. to 200° C., preferably from 40 to 120° C. and more preferably from 60° C. to 90° C.

The reaction time may, for example, be from 0.2 h to 72 hours, preferably from 1 to 36 h and most preferably from 2 to 10 h.

In a preferred embodiment of the process according to the invention, acid or acidic salt, water, the catalyst and the compounds of the formula (IV) are initially charged, the reaction mixture is placed under hydrogen pressure, preferably under a hydrogen pressure of about 1 bar, and the mixture is heated, for example within from 30 minutes to 2 hours, to reaction temperature. Once the reaction temperature is attained, the hydrogen pressure is increased to the desired value and, in a more preferred embodiment, is kept constant up to complete conversion.

Alternatively, the compound of the formula (IV) can also be added to the reaction mixture by pumping.

It is advantageous in the workup to initially filter off the catalyst and subsequently extract the reaction solution with an organic solvent, for example toluene. The extract is freed of solvent and the residue purified via distillation or recrystallization.

In the manner according to the invention, compounds of the formula (I) are obtained.

The process according to the invention is especially suitable for the preparation of 3-trifluoromethylbenzaldehyde, 3,5-bis(trifluoromethyl)benzaldehyde, 4-chlorobenzaldehyde and 3,5-difluorobenzaldehyde.

The compounds of the formula (I) prepared according to the invention, preferably without intermediate isolation, are reduced with hydrogen and in the presence of a catalyst to the compounds of the formula (V) where, in formula (V)

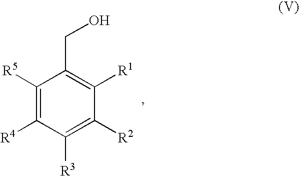

R¹, R², R³, R⁴ and R⁵ are each as defined under formula (I), including the areas of preference specified.

For example, this may be effected after the preparation according to the invention of the compounds of the formula (IV) by adding Raney nickel to the reaction solution and reducing under a hydrogen pressure of from 5 to 200 bar, preferably from 20 to 100 bar, or initially extracting the benzaldehydes prepared according to the invention and subsequently reducing the organic phase, for example by adding a Raney nickel catalyst as described above.

The compounds of the formulae (IV) and (V) preparable according to the invention are suitable in particular in a process for preparing agrochemicals and pharmaceuticals.

The advantage of the process according to the invention lies in its ease of performability and the saving of chemical process stages compared to the classical process for preparing benzaldehydes from benzonitriles.

The invention is further described by the following illustrative but non-limiting examples.

### EXAMPLES

### Example 1

An autoclave was initially charged with 10.8 g of 3,5-bis(trifluoromethyl)-benzonitrile in 70 g of 80% formic acid and also 0.9 g of Ni—Al-50/50 alloy. The autoclave was pressurized with 15 bar of nitrogen and heated to 70° C. Once the reaction temperature had been attained, the hydrogen pressure was increased to 3 bar and kept constant for 10 h. Subsequently, the mixture was cooled and filtered, the solution was extracted with toluene and subsequently fractionally distilled under reduced pressure. 8.8 g of 3,5-bis(trifluoromethyl)benzaldehyde were obtained (80% of theory).

### Example 2

An autoclave was initially charged with 7.7 g of 3-trifluoromethylbenzonitrile in 70 g of 70% formic acid and also 0.9 g of Ni—Al-50/50 alloy. The autoclave was pressurized with 15 bar of nitrogen and heated to 80° C. Once the reaction temperature had been attained, the hydrogen pressure was increased to 10 bar and kept constant for 7 h. Subsequently, the mixture was cooled and filtered, the solution was extracted with toluene and subsequently fractionally distilled under reduced pressure. 6.5 g of 3-trifluoromethylbenzaldehyde were obtained (85% of theory).

### Example 3

An autoclave was initially charged with 7.7 g of 3-trifluoromethylbenzonitrile in 50 g of 75% formic acid and also 1 g of NiFeCrAl prealloy. The autoclave was pressurized with 15 bar of nitrogen and heated to 80° C. Once the reaction temperature had been attained, the hydrogen pressure was increased to 6 bar and kept constant for 10 h. Subsequently, the mixture was cooled and filtered, the solution was extracted with toluene and subsequently fractionally distilled under reduced pressure. 7.0 g of 3-trifluoromethylbenzaldehyde were obtained (89% of theory).

### Example 4

An autoclave was initially charged with 6.3 g of 3,5-difluorobenzonitrile in 50 g of 75% formic acid and also 0.7 g of Ni—Al-50/50 alloy. The autoclave was pressurized with 15 bar of nitrogen and heated to 70° C. Once the reaction temperature had been attained, the hydrogen pressure was increased to 3 bar and kept constant for 10 h. Subsequently, the mixture was cooled and filtered, the solution was extracted with toluene and subsequently fractionally distilled under reduced pressure. 3.9 g of 3,5-difluorobenzaldehyde were obtained (61% of theory).

### Example 5

An autoclave was initially charged with 16.5 g of 4-chlorobenzonitrile in 139 g of 75% formic acid and also 1.4 g of Ni—Al-50/50 alloy. The autoclave was pressurized with 15 bar of nitrogen and heated to 70° C. Once the reaction temperature had been attained, the hydrogen pressure was increased to 3 bar and kept constant for 10 h. Subsequently, the mixture was cooled and filtered, the solution was extracted with toluene and subsequently fractionally distilled under reduced pressure. 15.1 g of 4-chlorobenzaldehyde were obtained (89% of theory).

### Example 6

An autoclave was initially charged with 14.5 g of 2-fluorobenzonitrile in 122 g of 75% formic acid and also 1.2 g of Ni—Al-50/50 alloy. The autoclave was pressurized with 15 bar of nitrogen and heated to 70° C. Once the reaction temperature had been attained, the hydrogen pressure was increased to 3 bar and kept constant for 10 h. Subsequently, the mixture was cooled and filtered, the solution was extracted with toluene and subsequently fractionally distilled under reduced pressure. 12.0 g of 2-fluorobenzaldehyde were obtained (80% of theory).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for preparing compounds of the formula (I) 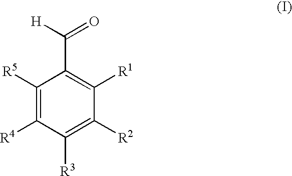
where
R¹, R², R⁴ and R⁵ are each independently hydrogen, fluorine, free or protected formyl, C₁-C₁₂-alkyl, C₁-C₁₂-alkoxy, C₁-C₁₂-haloalkyl, C₁-C₁₂-haloalkoxy, C₄-C₁₄-aryl, C₅-C₁₅-arylalkyl, —PO—[(C₁-C₈)-alkyl]₂, —PO—[(C₄-C₁₄)-aryl]₂, —PO—[(C₁-C₈)-alkyl)(C₄-C₁₄)-aryl)], tri(C₁-C₈-alkyl)siloxyl
or radicals of the formula (II)
A-CO-B (II)
where, each independently,
A is absent or is a C₁-C₈-alkylene radical and
B is R⁶, OR⁶, NHR⁷ or N(R⁷)₂,
where R⁶ is C₁-C₈-alkyl, C₅-C₁₅-arylalkyl, C₁-C₈-haloalkyl or C₄-C₁₄-aryl and
R⁷ is in each case independently C₁-C₈-alkyl, C₅-C₁₅-arylalkyl or C₅-C₁₄-aryl, or N(R⁷)₂ together is a cyclic amino radical,
or radicals of the formulae (IIIa-e)
A—E (IIIa)
A—SO₂—B (IIIb)
A—SO₂R⁶ (IIIc)
A—SO₃W (IIId)
A—COW (IIIe)
where
A, B and R⁶ are as defined above and
W is OH or NH₂, and
R³ is hydrogen, fluorine, chlorine or bromine,
comprising reacting compounds of the formula (IV) 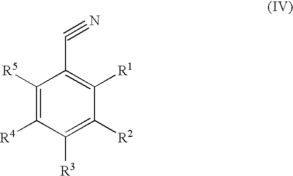
where
R¹, R², R³, R⁴ and R⁵ are as defined under formula (I)
with hydrogen, in the presence of water and in the presence of acid or acidic salts, the acids or acidic salts having a pK_{A} value of from 1 to 6 based on an aqueous basis system at 25° C. and in the presence of a nickel- and aluminium-containing catalyst.

2. Process according to claim 1, characterized in that R¹, R², R⁴ and R⁵ are each independently hydrogen, fluorine, C₁-C₄-alkyl, C₁-C₄-alkoxy, C₁-C₄-haloalkyl, C₁-C₄-haloalkoxy or N(R⁷)₂ where R⁷ is in each case independently, identically methyl or ethyl.

3. Process according to claim 1, characterized in that the compounds of the formula (IV) used are 4-chlorobenzonitrile, 3-trifluoro-methylbenzonitrile, 3,5-bis(trifluoromethyl)benzonitrile or 3,5-difluorobenzonitrile.

4. Process according to claim 1, characterized in that the weight ratio of acid and/or acidic salt to water is from 1:10 to 10:1.

5. Process according to claim 1, characterized in that the acids used are carboxylic acids.

6. Process according to claim 1, characterized in that the nickel- and aluminium-containing catalyst is used as a mixture of the metals or in the form of an alloy.

7. Process according to claim 1, characterized in that the nickel- and aluminium-containing catalyst comprises one or more metals from the group of chromium, rhenium, iron, cobalt, molybdenum and copper.

8. Process according to claim 1, characterized in that the amount of the nickel- and aluminium-containing catalyst is from 0.5 to 50 per cent by weight, based on the compound of the formula (IV) used.

9. Process according to claim 1, characterized in that the hydrogen pressure is from 0.2 to 100 bar.

10. Process according to claim 1, characterized in that the reaction temperature is from 20° C. to 200° C.

11. Process according to claim 1, characterized in that acid or acidic salt, water, catalyst and the compound of the formula (IV) are initially charged, the reaction mixture is placed under hydrogen pressure and heated to reaction temperature, before the hydrogen pressure is increased to the desired value.

12. Process according to claim 1, characterized in that, in a subsequent step, the compounds of the formula (I) are reduced with hydrogen and in the presence of a catalyst to compounds of the formula (V) where, in formula (V) 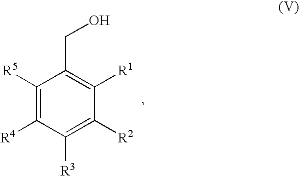
R¹, R², R³, R⁴ and R⁵ are each as defined under formula (I).

13. Process according to claim 12, characterized in that the reduction to compounds of the formula (V) is effected without intermediate isolation.

14. A process for preparing agrochemicals and pharmaceuticals comprising providing compounds of claim 1.
